(19) United States
(12) Patent Application Publication
(10) Pub. No.: US 2018/0044596 A1
(43) Pub. Date: Feb. 15, 2018

(54) PARTICULATE INORGANIC MATERIAL FOR HYDROLYSIS OF BIOMASS MATERIALS AND METHODS

(71) Applicant: IMERYS PIGMENTS, INC., Roswell, GA (US)

(72) Inventors: Anthony LYONS, Macon, GA (US); Chinmay PESHAVE, Milledgeville, GA (US); Kevin JONES, Alpharetta, GA (US)

(21) Appl. No.: 15/551,466

(22) PCT Filed: Feb. 10, 2016
(86) PCT No.: PCT/US16/17265

§ 371 Date: Aug. 16, 2017

Related U.S. Application Data
(60) Provisional application No. 62/117,285, filed on Feb. 17, 2015.

(51) Int. Cl. C10G 1/08; C01B 33/22; B01J 21/16; C01B 33/42; C13K 1/04; C13K 11/00; C07H 1/08; C01B 33/26

(52) U.S. Cl. CPC C10G 1/086; C07H 1/08; C01B 33/22; C01B 33/283; C01B 33/42; C13K 1/04; C13K 11/00; B01J 21/16; C01P 2004/60; C10G 2300/1011

(57) ABSTRACT

A composition comprising an inorganic particulate material adapted to convert a biomass material into hydrolysis products is provided. The inorganic particulate material comprises at least 0.1 wt. % of at least one impurity in its crystal structure based on the total weight of the inorganic particulate material. Methods of convert a biomass material into hydrolysis products using the compositions comprising an inorganic particulate material are also provided.

### CLAIM OF PRIORITY

This PCT International Application claims the benefit of priority of U.S. Provisional Application No. 62/117,285, filed Feb. 17, 2015, the subject matter of which is incorporated herein by reference in its entirety.

### FIELD OF THE DISCLOSURE

The present disclosure relates to particulate inorganic materials suitable for use in conversion of biomass materials to hydrolysis products. The present disclosure also relates to methods for converting biomass materials into hydrolysis products using particulate inorganic materials.

### BACKGROUND OF THE DISCLOSURE

Particulate inorganic materials such as phyllosilicates have a number of uses. For instance, kaolin may be used to solubilize, via hydrolysis, cellulosic materials at least in part. Such a hydrolysis process may be carried out by providing a mixture of kaolin, milling media, and cellulosic material and agitating the mixture in a ball, roller, jar, hammer, or shaker mill. However, previous processes may not produce fermentable products, for Instance, at acceptable yields for use industrially to provide precursors for biofuels.

Therefore, it may be desirable to provide compositions and methods for providing fermentable products from hydrolysis of biomass materials.

### SUMMARY

In the following description, certain aspects and embodiments will become evident. It should be understood that the aspects and embodiments, in their broadest sense, could be practiced without having one or more features of these aspects and embodiments. It should be understood that these aspects and embodiments are merely exemplary.

According to one aspect of this disclosure, a composition comprising an inorganic particulate material adapted to convert a biomass material into hydrolysis products is provided. The inorganic particulate material comprises at least 0.1 wt. % of at least one impurity in its crystal structure based on the total weight of the inorganic particulate material.

According to a second aspect of this disclosure, a method converting a biomass material into hydrolysis products is provided. The method comprises contacting the biomass material with an inorganic particulate material to form a feed and applying energy to the feed in an amount less than or equal to 50,000 kWh/DUST hydrolysis products to convert the biomass material to hydrolysis products.

It is to be understood that both the foregoing general description and the following detailed description are exemplary and explanatory only and are not restrictive of the invention, as claimed.

### BRIEF DESCRIPTION OF THE DRAWINGS

FIG. 1 shows a pareto chart showing the influence of titanium content of the clay, iron content of the clay, and both on the solubility of hydrolysis products produced using inorganic particulate clay materials having various titanium dioxide and iron oxide contents according to certain embodiments of the present invention.

FIG. 2 shows a plot of the solubility of hydrolysis products produced using inorganic particulate clay materials having various moisture contents according to certain embodiments of the present invention.

FIG. 3 shows a plot of the solubility of hydrolysis products produced using inorganic particulate clay materials processed at different starting temperatures according to certain embodiments of the present invention.

FIG. 4 shows a plot of the solubility of hydrolysis products produced using various amounts of acids in exemplary processes according to the present invention.

FIG. 5 shows a plot of the solubility of hydrolysis products produced using various energy inputs in exemplary processes according to the present invention.

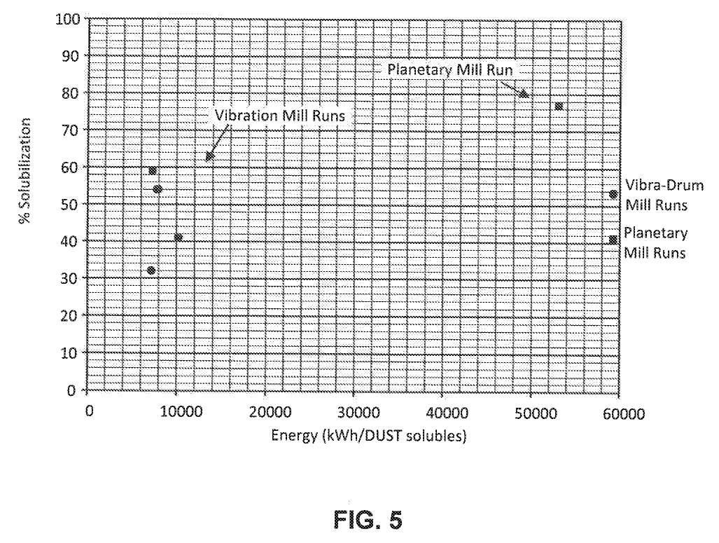

### DESCRIPTION OF EXEMPLARY EMBODIMENTS

Reference will now be made in detail to exemplary embodiments.

“Hydrolysis product,” as used herein, refers to any materials (e.g., soluble sugars, fermentable sugars, polysaccharide oligomer materials, cellobiose, glucose, fructose, levoglucosan, levoglucosenone, furfural, 5-hydroxymethylfurural, or combinations thereof) resulting from the breaking of the bonds between monomers (e.g., glucose monomers) of the biomass materials.

“Impurity,” as used herein, refers to a substance different from the primary composition of the inorganic particulate material and that may be present in the crystal structure of the inorganic particulate material and/or present outside of the crystal structure of the inorganic particulate material, for instance, in a mixture, aggregate, or like composition of the inorganic particulate material.

Particle size characteristics described herein are measured via sedimentation of the particulate material in a fully dispersed condition in an aqueous medium using a Sedigraph 5100 particle size analyzer supplied by Micrometrics Instruments Corporation Norcross, Ga., USA. The Sedigraph 5100 provides measurements and a plot of the cumulative percentage by weight of particles having a size referred to in the art as the “equivalent spherical diameter” or “esd.”

The term “d₅₀,” as used herein refers, to the median particle diameter and is the particle diameter at which 50% by weight of the product is smaller, and 50% by weight is larger, than the specified diameter.

The term “do,” as used herein, refers to the median particle diameter and is the particle diameter at which 90% by weight of the product is smaller, and 10% by weight is larger, than the specified diameter.

“Shape factor,” as used herein, is a measure of the ratio of particle diameter to particle thickness for a population of particles of varying size and shape. Shape factor may be measured using the electrical conductivity methods, apparatuses, and equations described in U.S. Pat. No. 5,576,617. As the technique for determining shape factor is further described in U.S. Pat. No. 5,576,617, the electrical conductivity of a composition of an aqueous suspension of orientated particles under test is measured as the composition flows through a vessel. Measurements of the electrical conductivity are taken along one direction of the vessel and along another direction of the vessel transverse to the first direction. Using the difference between the two conductivity measurements, the shape factor of the particulate material under test is determined. As generally used herein, the term “platy” refers to a material having a shape factor greater than or equal to 50.

“Steepness,” as used herein, refers to an indication of the particle size distribution monodispersity determined by the following formula:

$$Steepness = 100 \times \frac{{d}_{30}}{{d}_{70}}$$

dₓ is the equivalent spherical diameter relative to which x % by weight of the particles are finer.

### Inorganic Particulate Feed Material

According to a first aspect of the present invention, a composition is provided for converting biomass material into hydrolysis products. In certain embodiments, the composition comprise an inorganic particulate material having at least 0.1 wt. % of at least one impurity in its crystal structure based on the total weight of the inorganic particulate material, or at least 0.2 wt. % of at least one impurity in its crystal structure based on the total weight of the inorganic particulate material, or at least 1 wt. % of at least one impurity in its crystal structure based on the total weight of the inorganic particulate material. In other embodiments, the composition comprise an inorganic particulate material having less than or equal to 0.1 wt. % of at least one impurity in its crystal structure based on the total weight of the inorganic particulate material, or less than or equal to 1 wt. % of at least one impurity in its crystal structure based on the total weight of the inorganic particulate material.

For instance, the inorganic particulate material may comprise at least one impurity selected from the group consisting of titanium dioxide, iron oxide, and combinations thereof. In certain embodiments, the impurity is titanium dioxide present in an amount of at least 0.1 wt. % based on the total weight of the inorganic particulate material, or at least 0.5 wt. % based on the total weight of the inorganic particulate material, or at least 1 wt. % based on the total weight of the inorganic particulate material, or at least 1.5 wt. % based on the total weight of the inorganic particulate, or at least 2 wt. % based on the total weight of the inorganic particulate.

In certain embodiments, the inorganic particulate material comprises a combined titanium dioxide and iron oxide content of at least 2% based on the total weight of the inorganic particulate material, or at least 2.5 wt. % based on the total weight of the inorganic particulate material, or at least 3 wt. % based on the total weight of the inorganic particulate

In other embodiments, the inorganic particulate material has a moisture content of at least 0.5% based on the total weight of the inorganic particulate material, or at least 1 wt. % based on the total weight of the inorganic particulate material, or at least 1.5 wt. % based on the total weight of the inorganic particulate, or at least 2 wt. % based on the total weight of the inorganic particulate. In other embodiments, the inorganic particulate material has a moisture content ranging from 0.05% to 3.99% based on the total weight of the inorganic particulate material, or 0.05%% to 2.0% based on the total weight of the inorganic particulate material.

The inorganic particulate material used in certain embodiments of the present invention may be selected from, talc, mica, bentonite, vermiculite, halloysite, attapulgite, montmorillonite, illite, nacrite, dickite, and anauxite, or zeolites such as analcime, chabazite, heulandite, natrolite, phillipsite, stilbite, other clays, other phyllosilicates, and any inorganic particulate material having the general formula Al₂O₃.xSiO₂.nH₂O. In certain embodiments, the inorganic particulate material may comprise an anhydrous form of the aforementioned inorganic particulate materials. In one embodiment, the inorganic particulate material may comprise an acid. For example, the inorganic particulate material may comprise a superacid or citric acid.

In certain embodiments, the inorganic particulate material has at least 2 micromoles of acid sites per gram of the inorganic particulate material, or at least 3 micromoles of acid sites per gram of the Inorganic particulate material, or at least 4 micromoles of acid sites per gram of the inorganic particulate material measured using the pyridine adsorption test at 150° C. as described in Copeland, J. et al., “Surface Interactions of C₂ and C₃ Polyols with γ-Al₂O₃ and the Role of Coadsorbed Water”, Lanamuir, 29, p. 581-593, (2013).

Kaolin, also referred to as kaolin clay, china clay, or hydrous kaolin, contains predominantly the mineral kaolinite, together with small concentrations of various other minerals. Kaolinite may also be generally described as an aluminosilicate, aluminosilicate clay, or hydrous aluminosilicate (Al₂Si₂O₅(OH)₄). Kaolin clays were formed in geological times by the weathering of the feldspar component of granite. Primary kaolin clays are those which are found in deposits at the site at which they were formed, such as those obtained from deposits in South West England, France, Germany, Spain, and the Czech Republic. Sedimentary kaolin clays are those which were flushed out from the granite matrix at their formation site and were deposited in an area remote from their formation site, such as in a basin formed in the surrounding strata.

Talc is an oleophilic mineral composed of hydrated magnesium silicate generally having the chemical formula H₂Mg₃(SiO₃)₄ or Mg₃Si₄O₁₀(OH)₂. According to some embodiments, talc may also be chemically described by one or more of the following formulas: (Si₂O₅)₂Mg₃(OH)₂, Si₈Mg₆O₂₀(OH)₄, or Mg₁₂Si₁₈O₄₀(OH)₈. In certain embodiments, the talc may include impurities, which can include inorganics, such as carbonates, other magnesium silicates, iron compounds, and various organic materials that may be present. The impurities found in talcs may vary as to type and amount depending on the geographic source of the talc. There may also be minor elemental substitution of Mg with Fe, Al, or other elements in the crystalline structure of talc.

Talc may be characterized as being either microcrystalline or macrocrystalline in nature. In particular, talc may generally be in the form of individual platelets. The individual platelet size of the talc (e.g., the median particle diameter as measured by the Sedigraph method) of an individual talc platelet (a few thousand elementary sheets) may vary from approximately 1 micron to over 100 microns, depending on the conditions of formation of the talc deposit. Generally speaking, microcrystalline talc has small crystals, which provide a compact, dense ore. Macrocrystalline talc has large crystals in papery layers. In a microcrystalline structure, talc elementary particles are composed of small plates as compared to macrocrystalline structures, which are composed of larger plates.

In one embodiment, a single inorganic particulate material is used in order to hydrolyze a biomass material to form fermentable products. For example the single inorganic particulate material may be kaolin or the single inorganic particulate material may be talc. In another embodiment, a mixture of two or more phyllosilicate minerals may be milled together, or co-ground, with biomass materials to form hydrolysis products. For example, a mixture of kaolin and talc may be co-ground with cellulosic materials using the method of certain embodiments of the invention.

The shape factor of the “feed” phyllosilicate mineral (e.g., kaolin, talc, mica, and/or bentonite) may ranging from 1 to 100, for example less than 90, or less than 80, or less than 70, or less than 60, or less than 50, or less than 40, or less than 30, or less than 20, or less than 15. The shape factor of the feed phyllosilicate mineral may be greater than 10, or may be greater than 20, or may be greater than 30, or may be greater than 40, or may be greater than 50 or may be greater than 60, or may be greater than 70 or may be greater than 80. In one embodiment, the feed mineral is talc having a shape factor of from 10 to 45, or from 15 to 35. In another embodiment, the feed mineral is a kaolin having a shape factor of from 10 to 50, or a shape factor of from 2 to 40.

Where kaolin is present in the feed, it may have a d₅₀ in the range of from 0.1 to 20 μm, for example in the range of from 0.1 to 10 μm, for example in the range of from 0.1 to 5 μm. The steepness value of kaolin used as the feed mineral may be in the range of from 10 to 50.

Where talc is present in the feed, it may have a d₅₀ in the range of from 2 to 20 μm, for example in the range of from 2 to 15 μm, for example in the range of from 2 to 10 μm. The steepness value of talc used as the feed mineral may be in the range of from 15 to 40, for example from 25 to 35.

According to some embodiments, the talc may be a microcrystalline talc. According to some embodiments, the talc may be a macrocrystalline talc.

The inorganic particulate material may have a particle size distribution such that 100% by weight of the particles are smaller than 2 μm, or no more than 99% by weight of the particles are smaller than 2 μm, or no more than 95% by weight of the particles are smaller than 2 μm. In an embodiment the inorganic particulate material may have a particle size distribution such that no more than 80% by weight of the particles are smaller than 2 μm. In another embodiment, the inorganic particulate material may have a particle size distribution such that no more than 70% by weight of the particles are smaller than 2 μm. In another embodiment, the inorganic particulate material may have a particle size distribution such that no more than 60% by weight of the particles are smaller than 2 μm. In yet another embodiment, the inorganic particulate material may have a particle size distribution such that no more than 50% by weight of the particles are smaller than 2 μm. In an embodiment the inorganic particulate material may have a particle size distribution such that no more than 40% by weight of the particles are smaller than 2 μm. In another embodiment, the inorganic particulate material may have a particle size distribution such that no more than 35% by weight of the particles are smaller than 2 μm. In another embodiment, the inorganic particulate material may have a particle size distribution such that no more than 30% by weight of the particles are smaller than 2 μm. In yet another embodiment, the inorganic particulate material may have a particle size distribution such that no more than 25% by weight of the particles are smaller than 2 μm. In one embodiment, the inorganic particulate material may have a particle size distribution such that no more than 20% by weight of the particles are smaller than 2 μm. In certain embodiments, the inorganic particulate material may have a particle size distribution such that no more than 15% by weight of the particles are smaller than 2 μm.

According to some embodiments, the inorganic particulate material may have a d₉₀ less than or equal to 20 microns, such as, for example, less than or equal to 15 microns, less than or equal to 10 microns, less than or equal to 7 microns, less than or equal to 5 microns, less than or equal to 4 microns, less than or equal to 3 microns, or less than or equal to 2 microns.

In an embodiment of the invention, the only particulate mineral present in the aqueous suspension is the phyllosilicate mineral.

The inorganic particulate material for use as feed material may be prepared from the raw natural material by one or more pre-processing steps. For example, the raw material may be processed in aqueous suspension to remove contaminants and impurities, for example by magnetic separation. The raw material may also be bleached using methods known to those skilled in the art. The raw material may also be subjected to a preliminary process to reduce the particle size of the agglomerated raw material. For example, the raw material may be ground or milled to reduce the particle size to the desired feed material particle size. In certain embodiments where the phyllosilicate mineral is talc, the feed material may be subjected to an initial dry grinding step. In certain embodiments where the phyllosilicate mineral is kaolin, the feed material may be subjected to an initial wet grinding step.

The suspension comprising the coarse, pre-processed material may then be dewatered by, for example, use of a tube press, although other methods of dewatering are also contemplated, such as thermal or spray drying. In certain embodiments, the dewatered product may have a suitable high solids content corresponding to that desired for the grinding stage.

In alternate embodiments, the dewatered product may be dispersed using a suitable dispersing agent.

Suitable dispersing agents are chemical additives capable, when present in a sufficient amount, of acting on the particles of the particulate material to prevent or effectively restrict flocculation or agglomeration of the particles to a desired extent, according to normal processing requirements. The dispersant may be present in levels up to 1% by weight, and includes, for example, polyelectrolytes such as polyacrylates and copolymers containing polyacrylate species, especially polyacrylate salts (e.g., sodium and aluminium optionally with a group II metal salt), sodium hexametaphosphates, non-ionic polyol, polyphosphoric acid, condensed sodium phosphate, non-ionic surfactants, alkanolamine and other reagents commonly used for this function. The dispersant may, for example, be selected from conventional dispersant materials commonly used in the processing and grinding of inorganic particulate materials. Such dispersants will be well recognized by those skilled in this art. They are generally water-soluble salts capable of supplying anionic species which in their effective amounts can adsorb on the surface of the inorganic particles and thereby inhibit aggregation of the particles. The unsolvated salts suitably include alkali metal cations such as sodium. Solvation may in some cases be assisted by making the aqueous suspension slightly alkaline. Examples of suitable dispersants include: water soluble condensed phosphates, e.g., polymetaphosphate salts [general form of the sodium salts: (NaPO3)x] such as tetrasodium metaphosphate or so-called “sodium hexametaphosphate” (Graham's salt); water-soluble salts of polysilicic acids; polyelectrolytes; salts of homopolymers or copolymers of acrylic acid or methacrylic acid, or salts of polymers of other derivatives of acrylic acid, suitably having a weight average molecular mass of less than 20,000. Sodium hexametaphosphate and sodium polyacrylate, the latter suitably having a weight average molecular mass in the range of 1,500 to 10,000, are especially preferred.

In alternate embodiments, the inorganic particulate material may comprise a crude inorganic particulate material that has not been beneficiated or that has been minimally beneficiated. For instance, the inorganic particulate feed material may not have been subjected to one or more of the following processes: drying, crushing, blunging, classification, ozone treatment, selective flocculation, magnetic separation, leaching, bleaching, and filtration. As such, in some embodiments, the inorganic particulate may be devoid of ions, sodium compounds, sulfates, and/or processing polymers. In other embodiments, the inorganic particulate material may be substantially devoid or devoid of any dispersants.

In certain embodiments the composition adapted to hydrolyze a biomass material may comprise an additive. For instance, the additive may be selected from the group consisting of citric acid, phosphoric acid, sulfuric acid, and combinations thereof. In certain embodiments, the additive may be present in an amount ranging from 0.1 wt. % to 20 wt. % based on the total weight of the composition. For instance, the additive may be present in an amount ranging from 1 wt. % to 20 wt %, or greater than or equal to than 1 wt. % based on the total weight of the composition.

In certain embodiments, the additive may comprise iron oxide in an amount ranging from 0.1 to 3% by weight based on the total weight of the composition.

### Biomass Feed Material

The inorganic particulate material used in certain embodiments of the present invention may be selected from wood, paper, switchgrass, wheat straw, agricultural plants, trees, agricultural residues, herbaceous crops, starches, corn stover, saw dust, and high cellulose municipal, industrial solid wastes, any other cellulosic materials, and combinations thereof. In certain embodiments, the biomass material may comprise microcrystalline cellulose and/or wood flour.

According to certain embodiments, the biomass feed material may be present in the process feed in an amount ranging from 1 to 30% based on the total weight of the process feed.

### Hydrolysis Process

According to another aspect of the present invention, a method for converting a biomass material into hydrolysis products is provided. In certain embodiments, the method comprises contacting the biomass material with an inorganic particulate material to form a feed; and applying energy to the feed in an amount less than or equal to 50,000 kWh/dry US ton (DUST) hydrolysis products to convert the biomass material to hydrolysis products. For instance, the step of applying energy comprises applying energy to the feed in an amount less than or equal to 15,000 kWh/DUST.

In certain embodiments, the application of energy occurs at a temperature ranging from 25° C. to 180° C. In other embodiments, the application of energy occurs at a temperature ranging from 60° C. to 120° C. In particular embodiments, the process feed may be preconditioned by heating to a temperature ranging from 25° C. to 180° C. or from 60° C. to 120° C. or from 120° C. to 180° C.

In one embodiment, the method further comprises including milling media (e.g., steel ball media, carbon steel media, tungsten carbide media, and combinations thereof) into the feed. For instance, the method comprises providing at least two milling media in the feed, wherein a first milling media has a first size and a second milling media has a second size. The size of the milling media may range from 1 mm to 75 mm or from 4 mm to 35 mm.

In certain embodiments, the biomass material is converted to hydrolysis products that comprise at least 1% soluble hydrolysis products based on the total weight of the hydrolysis products. In other embodiments, the biomass is converted hydrolysis products that comprise at least 10% soluble hydrolysis products based on the total weight of the hydrolysis products. In other embodiments, the biomass is converted hydrolysis products that comprise at least 30% soluble hydrolysis products based on the total weight of the hydrolysis products. In other embodiments, the biomass is converted hydrolysis products that comprise at least 50% soluble hydrolysis products based on the total weight of the hydrolysis products.

According to some embodiments, the ratio of the inorganic particulate material to the biomass material may be in a range from 10:1 to 1:10 by weight or 25:75 to 75:25 by weight, such as, for example, from 30:70 to 70:30, from 40:60 to 60:40, from 45:55 to 55:45, from 20:80 to 50:50, from 50:50 to 80:20, from 20:80 to 40:60, or from 60:40 to 80:20 by weight. According to some embodiments, the ratio of talc to the second component in the additive may be 50:50 by weight.

In certain embodiments, the hydrolysis process may be carried out in attrition mill, a planetary mill, or a vibration mill grinder.

In certain embodiments, the hydrolysis process may comprise a sequence of grinding the kaolin and biomass mixture for 10 minutes to 10 hours, washing the material out of the reactor and then recycling the unconverted biomass to the reactor for more processing along with fresh material. In certain embodiments, the processing may be carried out for over 10 hours. In certain embodiments, the hydrolysis of cellulose has complex kinetics in which the glucose can be broken down or repolymerized as it is produced. Depending upon the reaction conditions the peak production of glucose from the reactor will likely be in the prescribed range of time.

### Example 1

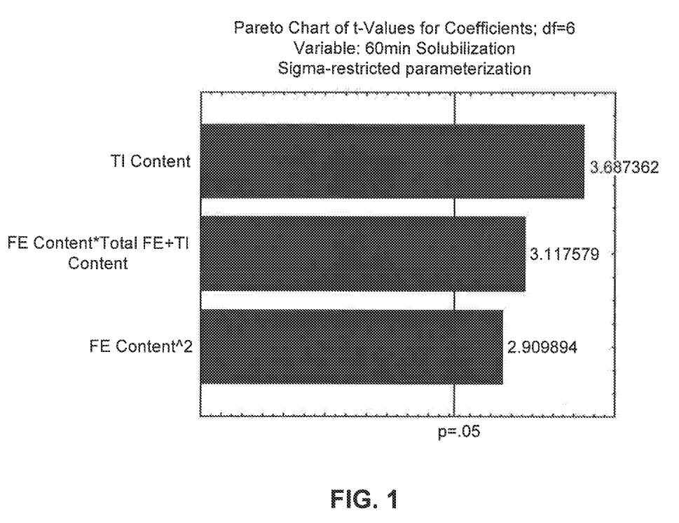

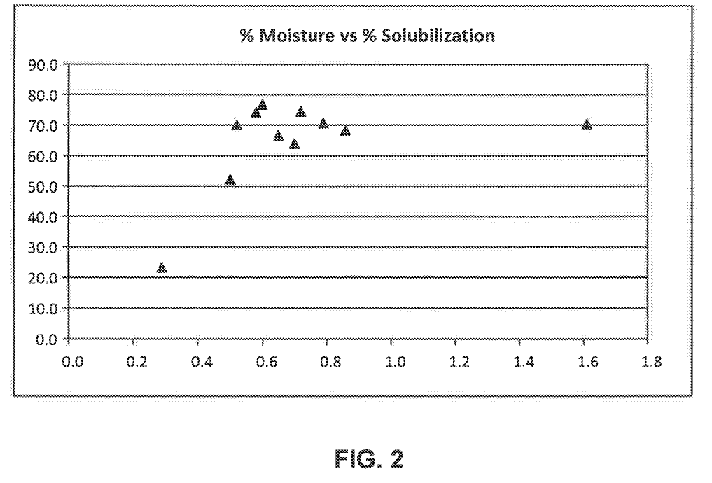

15 grams microcrystalline cellulose was hydrolyzed with 15 grams of each of the inorganic particulate materials listed in Table 1 below in a mill with three types of steel ball media. The three steel media having sizes of 19, 14, and 9 mm were used in amounts of 141 grams, 179 grams, and 61 grams, respectively. The percent (%) solubility of each resulting hydrolysis product was compared to various parameters of the samples and process. The pareto analysis of the % solubilization as a function of titanium content of the clay, iron content of the clay, and both are shown in FIG. 1. The percent solubilization as compared to the moisture content is shown in FIG. 2.

TABLE 1
Inorganic Particulate materials

| | | | | | Solubilization | Solubilization |
|---|---|---|---|---|---|---|
| | | Fe2O3 | TiO2 | Moisture | after 60 min | after 60 min |
| | Description | (wt. %) | (wt. %) | (wt. %) | (wt. %) | (wt. %) |
| Beneficiated | Airfloat clay | 1.68 | 1.39 | 1.6 | 31.0 | 70.4 |
| Clay 1 | with d50 = | | | | | |
| | 4.43 um | | | | | |
| Beneficiated | Delaminated | 0.39 | 0.79 | 0.5 | 14.4 | 52.2 |
| Clay 2 | kaolin with d50 = | | | | | |
| | 0.57 um | | | | | |
| Beneficiated | Fractionated | 0.62 | 1.09 | 0.8 | 24.3 | 70.7 |
| Clay 3 | kaolin with d50 = | | | | | |
| | 0.29 um | | | | | |
| Crude Clay 1 | Clay with d50 = | 1.21 | 3.02 | 0.7 | 36.5 | 74.5 |
| | 0.33 um | | | | | |
| Crude Clay 2 | Hard clay with | 0.62 | 1.83 | 0.6 | 36.1 | 76.7 |
| | d50 = 0.29 um | | | | | |
| | flocculated | | | | | |
| | with H2SO4 | | | | | |
| Crude Clay 3 | Crude with d50 = | 0.7 | 1.5 | 0.7 | 22.3 | 66.8 |
| | 1.46 um | | | | | |
| Crude Clay 4 | Crude with d50 = | 0.2 | 1.38 | 0.6 | 44.1 | 74.1 |
| | 2.21 um | | | | | |
| Crude Clay 5 | Hard clay | 0.67 | 1.76 | 0.5 | 52.5 | 70.1 |
| | crude with d50 = | | | | | |
| | 0.61 um | | | | | |
| Crude Clay 6 | Hard clay | 0.59 | 1.7 | 0.9 | 53.0 | 68.3 |
| | crude with d50 = | | | | | |
| | 0.4 um | | | | | |
| Crude Clay 7 | Crude with d50 = | 1.04 | 1.34 | | 21.4 | 64 |
| | 0.41 um | | | | | |
| Metakaolin | | | | 0.3 | 8.6 | 23.2 |

### Example 2

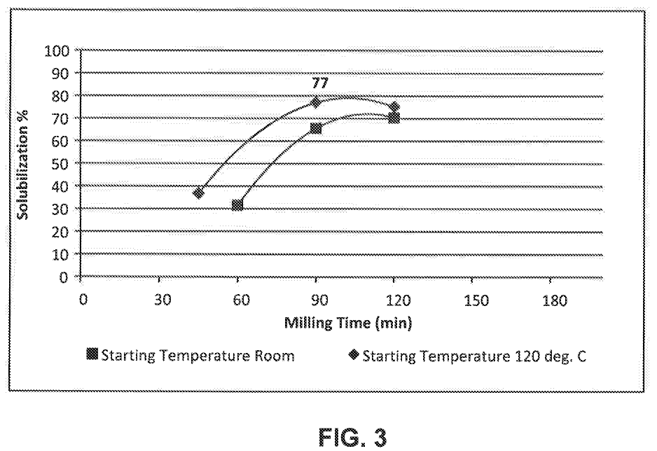

15 grams microcrystalline cellulose was hydrolyzed with 15 grams of the Beneficiated Clay 1 materials listed in Table 1 in a mill with three types of steel ball media. The three steel media having sizes of 19, 14, and 9 mm were used in amounts of 141 grams, 179 grams, and 61 grams, respectively. Two process conditions were tested: 1) a starting temperature of 25° C. and a clay moisture content of 2 wt. % 2) a starting temperature of 120° C. and a clay moisture content of 0.15%. The percent (%) solubility of each resulting hydrolysis products as a function of milling time is shown in FIG. 3.

### Example 3

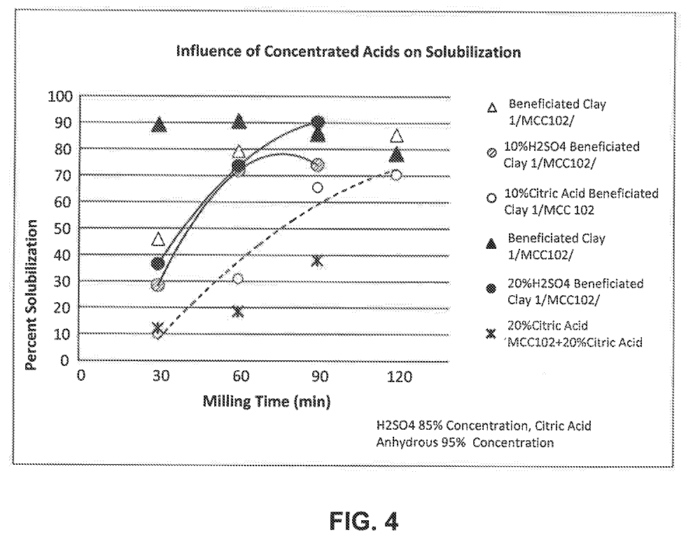

15 grams microcrystalline cellulose was hydrolyzed with 15 grams of the Beneficiated Clay 1 materials listed in Table 1 in a mill with three types of steel ball media. The three steel media having sizes of 19, 14, and 9 mm were used in amounts of 141 grams, 179 grams, and 61 grams, respectively. Citric acid anhydrous (>95% purity) or sulfuric acid (85% purity) were added in amounts of 10% and 20% based on the mass of the cellulosic material. The results showed that citric acid at 10% and 20% improved the solubilization of the hydrolysis products, as seen in FIG. 4

### Example 3

15 grams microcrystalline cellulose was hydrolyzed with 15 grams of the Beneficiated Clay 1 materials listed in Table 1 in a mill with three types of steel ball media. The three steel media having sizes of 19, 14, and 9 mm were used in amounts of 141 grams, 179 grams, and 61 grams, respectively. The starting temperature was 120° C. The grinding methods was carried out in a planetary mill. In another test, the same materials were ground in a Viba-drum mill with 75 pounds of media at the same ratios and a ratio of 28:1 of media to clay and cellulosic material, where the clay to cellulosic material ratio is 1:1.

In this study the active power (kW) was recorded and used for the energy calculations. The active power was monitored after every 15 minutes.

$$P = {\sum }_{i = 1}^{m} ( {P}_{i} )$$

where i is the number of hours the milling process was carried out. The energy consumption was calculated by reducing the active power P (kW) recorded in presence of clay-cellulose mixture by the idle power P₀ (kW) (without grinding media or mixture) at respective grinding process conditions. The energy consumption E [kWh/DUST] was determined by,

$$E = \frac{P - {P}_{0}}{M} \frac{[ kWh ]}{[ DUST ]}$$

where M is the mass of clay-cellulose mixture in dry US tons.

The planetary mill total power consumption was 0.675 KW. Thus, the total power consumption=0.675 kW/3.31E-05 DUST=20,412 kWh/DUST based on total mixture. Thus, based on cellulosic material, the energy utilization is 20,412*2=40,824 kWh/DUST. Out of the total amount of cellulosic milled, 77% was converted to water soluble products. Thus, [100*40,824]/77=53,018 kWh/DUST.

The vibration mill with two motors utilized total 8.063 kWh power during the grinding trial. Thus, based on cellulose, the energy utilization is 2150*2=4300 kWh/DUST. Out of the total amount of cellulose milled, 59% was converted to water soluble products. Thus, [100*4300]/59=7,288 kWh/DUST

Other embodiments will be apparent to those skilled in the art from consideration of the specification and practice of the exemplary embodiments disclosed herein. It is intended that the specification and examples be considered as exemplary only, with a true scope and spirit of the invention being indicated by the following claims.

1. A composition comprising:
an inorganic particulate material adapted to convert a biomass material into hydrolysis products,
wherein the inorganic particulate material comprises at least 0.1 wt. % of at least one impurity in its crystal structure based on the total weight of the inorganic particulate material.

2. The composition of claim 1, wherein the at least one impurity is selected from the group consisting of titanium dioxide, iron oxide, and combinations thereof.

3. The composition of claim 1, wherein the at least one impurity is titanium dioxide present in an amount of at least 0.1 wt. % based on the total weight of the inorganic particulate material.

4. The composition of claim 1, wherein the inorganic particulate material comprises has a moisture content of at least 0.05% based on the total weight of the inorganic particulate material.

5. The composition of claim 1, wherein the inorganic particulate material is selected from the group consisting of kaolin, talc, mica, and bentonite.

6. The composition of claim 1, wherein the inorganic particulate material has at least 2 micromoles of acid sites per gram of the inorganic particulate material at 150° C.

7. The composition of claim 1, further comprising at least one additive.

8. The composition of claim 7, wherein the additive is selected from the group consisting of citric acid, iron oxide, and combinations thereof.

9. The composition of claim 7, wherein the additive is present in an amount ranging from 0.1 wt. % to 20.0 wt. % based on the total weight of the composition.

10. The composition of claim 1, wherein the inorganic particulate material is substantially devoid of dispersants.

11. A method for converting a biomass material into hydrolysis products comprising:
contacting the biomass material with an inorganic particulate material to form a feed; and
applying energy to the feed in an amount less than or equal to 50,000 kWh/DUST hydrolysis products to convert the biomass material to hydrolysis products.

12. The method of claim 11, wherein the step of applying energy comprises applying energy to the feed in an amount less than or equal to 15,000 kWh/DUST.

13. The method of claim 11, wherein the application of energy occurs at a temperature ranging from 25° C. to 180° C.

14. The method of claim 11, further comprising providing at least two milling media in the feed, wherein a first milling media has a first size and a second milling media has a second size.

15. The method of claim 11, wherein the biomass material is converted to hydrolysis products that comprise at least 1% soluble hydrolysis products based on the total weight of the hydrolysis products.

16. The method of claim 11, wherein the biomass is converted hydrolysis products that comprise at least 30% soluble hydrolysis products based on the total weight of the hydrolysis products.

17. The method of claim 11, comprising providing at least one additive in the feed.

18. The method of claim 17, wherein the additive is selected from the group consisting of citric acid, iron oxide, and combinations thereof.

19. The method of claim 17, wherein the additive is present in an amount ranging from 0.1 wt. % to 20.0 wt. % based on the total weight of the inorganic particulate material.

20. The method of claim 11, wherein the inorganic particulate material is substantially devoid of dispersants.
